(19) United States
(12) Patent Application Publication
(10) Pub. No.: US 2021/0254010 A1
(43) Pub. Date: Aug. 19, 2021

(54) Isolation of Mesenchymal Stromal Cells

(71) Applicant: Abraham J and Phyllis Katz Cord Blood Foundation, Cleveland, OH (US)

(72) Inventors: Mary Laughlin, Cleveland, OH (US); Jeong-Su Do, Cleveland, OH (US); Wouter Van't Hof, Cleveland, OH (US)

(21) Appl. No.: 16/973,220

(22) PCT Filed: May 31, 2019
(86) PCT No.: PCT/US19/34789

§ 371 Date: Dec. 8, 2020

Related U.S. Application Data
(60) Provisional application No. 62/684,854, filed on Jun. 14, 2018.

(51) Int. Cl. C12N 5/0775; B01D 21/26

(52) U.S. Cl. CPC C12N 5/0665; B01D 21/262; B01D 2251/70; C12N 2500/84; B01D 2251/90; C12N 2502/1157

(57) ABSTRACT

Methods for isolating mesenchymal stromal cells from umbilical cord blood. The methods include providing a blood collection bag having raw umbilical cord blood contained therein, draining the umbilical cord blood from the blood collection bag, introducing a cell detachment solution into the blood collection bag to dissociate mesenchymal stromal cells adhered to the bag surface, thereby forming a mesenchymal stromal cell enriched fluid, and voiding the mesenchymal stromal cell enriched fluid into a collection apparatus for further processing and culturing.

### CROSS REFERENCE TO RELATED APPLICATIONS

This application claims the priority benefit of U.S. Provisional Application No. 62/684,854, filed Jun. 14, 2018, which application is incorporated herein by reference.

### FIELD OF THE INVENTION

The present disclosure relates generally to isolating mesenchymal stromal cells and, more particularly, methods for isolating mesenchymal stromal cells from umbilical cord blood.

### BACKGROUND

Mesenchymal stromal cells (MSCs), sometimes referred to as mesenchymal stem cells, have been studied extensively because of their high potential for multi-lineage differentiation and proliferation, characteristics that make the cells useful for therapeutic purposes. Historically, MSCs have been derived from sources such as bone marrow (BM-MSCs) and adipose tissue (AT-MSCs). However, the use of BM-MSCs and AT-MSCs has disadvantages, including the necessity of invasive harvest of bone marrow and adipose tissue from patients or donors. Thus, alternate sources of MSCs have been intensively sought, including cells from human placental tissues (e g amnion, microvillus, Wharton's jelly and umbilical cord perivascular cells). These human placental tissues, however, currently require complex procedures for cell isolation including enzymatic digestion or prolonged culture when using a tissue explants method, and the procedures have low MSC isolation success rates.

It is well known that there are MSCs in human umbilical cord blood (CB-MSCs). Cord blood as a source for MSCs has several advantages over other MSC sources such as bone marrow and adipose tissue. Harvesting cord blood is relatively easy, less expensive, and non-invasive compared to aspirating bone marrow and adipose tissue, and cord blood banking systems have been developed worldwide meeting technical requirements for clinical grade cellular products.

In conventional procedures for isolating MSCs from cord blood, cord blood is collected from the umbilical cord after the delivery of placenta or while in utero in blood bags with an anticoagulant such as citrate phosphate dextrose. Then, mononuclear cells (MNCs) are separated from the cord blood by density gradient centrifugation, and are plated in a tissue culture apparatus. Non-adherent cells are then removed after a period of cell culture, typically days to weeks, and the adherent cells or individual colonies thereof are expanded to yield a population of isolated MSCs.

CB-MSCs have not yet achieved wide-spread experimental and clinical application because of their low frequency in cord blood and the inconsistency with which they are successfully isolated. Thus, the main factor limiting CB-MSC use in research and clinical applications are the problems arising from isolating CB-MSCs from cord blood. At present, the success rate of isolating MSCs from umbilical cord blood is on the order of 10-20%, meaning that only 1 or 2 out of 10 umbilical cord blood samples yield CB-MSCs. While selecting umbilical cord blood samples based on specific markers may improve the success rate of CB-MSC isolation to some extent, cord blood screening limits the utility of CB-MSC isolation to select patient populations and only marginally improves the success rate of MSC isolation from umbilical cord blood.

Therefore, what are needed are quick and efficient methods for isolating MSCs from cord blood with improved success rates that provide CB-MSCs in a sufficient amount for various research and clinical applications.

### SUMMARY OF THE INVENTION

Provided are methods for isolating MSCs from umbilical cord blood. In one aspect, a method for isolating mesenchymal stromal cells from umbilical cord blood is provided which includes: providing a polymer lined blood collection container having raw umbilical cord blood contained therein; draining the umbilical cord blood from the blood collection container; detaching mesenchymal stromal cells adhered to the blood collection container with a cell detachment solution introduced into the blood collection container to form a mesenchymal stromal cell enriched fluid; and voiding the mesenchymal stromal cell enriched fluid into a collection apparatus for further processing and culturing.

### BRIEF DESCRIPTION OF THE DRAWINGS

FIG. 1 shows a study design to test whether cells remaining in an umbilical cord blood (UCB) collection bag (termed “Bag Cells”) after draining blood therefrom may contain early mesenchymal stromal cell (MSC) precursors that would increase mesenchymal stromal cell output when added to mononuclear cells at initial plating.

FIG. 2 shows four different methods used to culture UCB derived MNCs and Bag Cells. The Twisha protocol cultures UCB derived MNCs and Bag Cells in Iscove's Modified Dulbecco's Medium (IMDM) with 20% human serum and fibroblast growth factor (FGF). The Basabi protocol cultures UCB derived MNCs and Bag Cells in Dulbecco's Modified Eagle's Medium (DMEM) with supplement (supplement is a human platelet rich plasma that serves as a cytokine rich replacement for fetal bovine serum, sold by Compass-Biomedical). The other two protocols culture UCB derived MNCs and Bag Cells in Dulbecco's Modified Eagle's Medium (DMEM) with 20% Fetal Bovine Serum (FBS), with or without dexamethasone (DEX). Dexamethasone (10 nM) was used to block monocytes adhering to the tissue culture flask.

FIG. 3 shows UCB derived MNCs and Bag Cells cultured according to the Twisha protocol at day 6. At day 6, some of the cells were attached to the culture flask; however, there was no clear MSC morphology.

FIG. 4 shows UCB derived MNCs and Bag Cells cultured according to the Twisha protocol at day 14. At day 14, some cells have MSC morphology but not many No significant difference in MSC outgrowth is seen between the MNC and MNC+Bag culture.

FIG. 5 shows UCB derived MNCs and Bag Cells cultured according to the Twisha protocol at day 20. At day 20, cells are not growing well.

FIG. 6 shows UCB derived MNCs and Bag Cells cultured according to the Basabi protocol at day 6. At day 6, cells with MSC morphology were detected.

FIG. 7 shows UCB derived MNCs and Bag Cells cultured according to the Basabi protocol at day 13. At day 13, cells are not growing well and many are dead.

FIG. 8 shows UCB derived MNCs and Bag Cells cultured according to the Basabi protocol at day 20. At day 20, many of the cells have died. Supplement does not appear to be great for UCB derived MSC.

FIG. 9 shows UCB derived MNCs and Bag Cells cultured according to a DMEM+20% FBS protocol at day 6. At day 6, cell growth looks similar comparing MNC alone vs. MNC+Bag cells.

FIG. 10 shows UCB derived MNCs and Bag Cells cultured according to a DMEM+20% FBS protocol at day 13. At day 13, many cells were detected in the MNC+Bag culture, and many cells have spindle shape MSC morphology in the MSC+Bag condition.

FIG. 11 shows UCB derived MNCs and Bag Cells cultured according to a DMEM+20% FBS protocol at day 20. At day 20, many cells have died in the MNC alone culture condition. Many cells have MSC morphology in MNC+Bag condition.

FIG. 12 shows UCB derived MNCs and Bag Cells cultured according to a DMEM+20% FBS+DEX protocol at day 6.

FIG. 13 shows UCB derived MNCs and Bag Cells cultured according to a

DMEM+20% FBS+DEX protocol at day 13. At day 13, the MNC alone condition lost many cells but the MNC+Bag condition still has many cells with MSC morphology.

FIG. 14 shows UCB derived MNCs and Bag Cells cultured according to a DMEM+20% FBS+DEX protocol at day 20. At day 20, the MNC alone condition significantly lost cells compared to MNC+Bag condition. MNC+Bag cells continue to change cell morphology to be consistent with MSC.

FIG. 15 shows UCB derived MNCs and Bag Cells cultured according to the Twisha protocol at week 12. At week 12, the Twisha protocol demonstrates improved MSC outgrowth at 12 weeks in the MNC+Bag condition compared with MNC alone. However, the rate of growth with this protocol is slow.

FIG. 16 shows flow cytometry data comparing MNC alone versus MNC+Bag. MSC are defined as CD44, CD90, CD73 and CD105 positive. Cells from MNC alone are CD44 and CD105 positive but at lower expression levels. Cells from MNC+Bag are CD44, CD105, and CD73 positive, consistent with defined MSC phenotype.

FIG. 17 shows UCB derived MNCs and Bag Cells cultured according to the Twisha protocol at day 7. At day 7, some cells were attached to the culture flask; however, there was no clear MSC morphology.

FIG. 18 shows UCB derived MNCs and Bag Cells cultured according to the Twisha protocol at day 21. At day 21, the MNC+Bag culture cells showed MSC morphology whereas the MNC alone culture did not show MSC outgrowth.

FIG. 19 shows UCB derived MNCs and Bag Cells cultured according to a DMEM+20% FBS protocol at day 7. At day 7, cell growth looks similar comparing MNC alone vs. MNC+Bag cells.

FIG. 20 shows UCB derived MNCs and Bag Cells cultured according to a DMEM+20% FBS protocol at day 21. At day 21, many cells have MSC morphology in the MNC alone and MNC+Bag conditions.

FIG. 21 shows UCB derived MNCs and Bag Cells cultured according to a DMEM+20% FBS protocol at day 28. At day 28, cells with MSC morphology are present in the MNC+Bag condition but not the MNC alone condition.

FIG. 22 shows cell counts for MNCs and Bag Cells, derived from several different donors, cultured according a Twisha protocol or a DMEM+20% FBS protocol.

The MNC+Bag culture condition has many more cells compared to the MNC alone culture condition.

FIG. 23 shows flow cytometry data from a phenotype analysis of a UCB derived MNC+Bag condition cultured according to the Twisha protocol at day 21. The MNC+Bag condition cultured according to the Twisha protocol at day 21 was positive for MSC positive-markers.

FIG. 24 shows flow cytometry data from a phenotype analysis of a UCB derived MNC+Bag condition cultured according to the Twisha protocol at day 21. The MNC+Bag condition cultured according to the Twisha protocol at day 21 was negative for MSC negative-markers.

FIG. 25 shows results from a T cell suppression assay that functionally tested for MSCs. Human PBMC isolated naive CD4 T cells were stimulated with Anti-CD3/Ab with APC. After 5 days of stimulation, CFSE intensity was analyzed.

FIG. 26 shows results from a mixed lymphocyte reaction assay that functionally tested for MSCs. Human PBMC isolated naive CD4 T cells were stimulated with allo stimulator. After 5 days stimulation, CFSE intensity was analyzed.

FIG. 27 shows sample collected for phenotype analysis and functional test using BD STEMLOW™ hMDC analysis kit and a suppression assay.

FIG. 28 shows the CB-MSC cell number count.

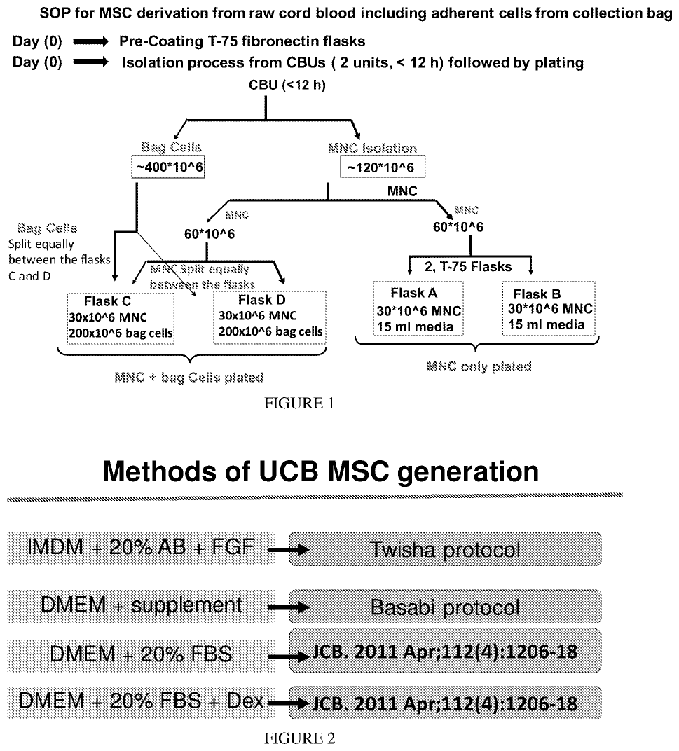

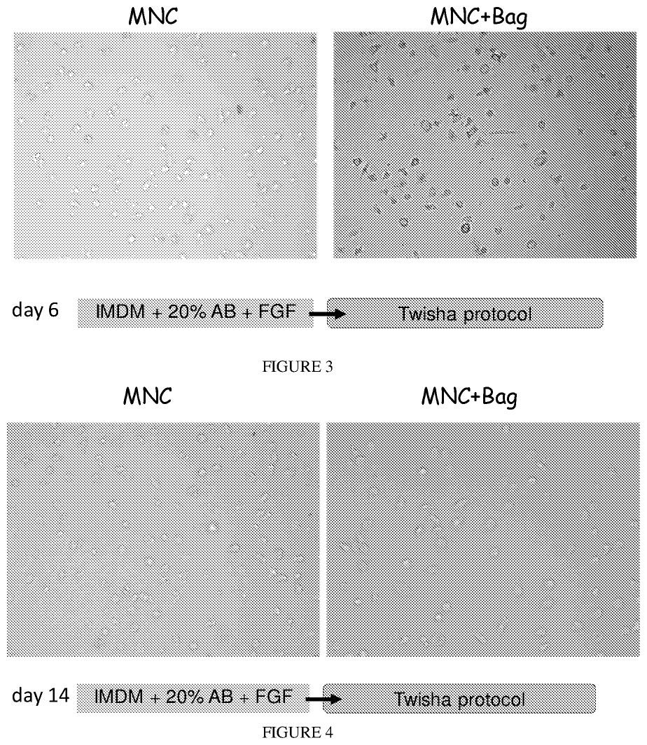

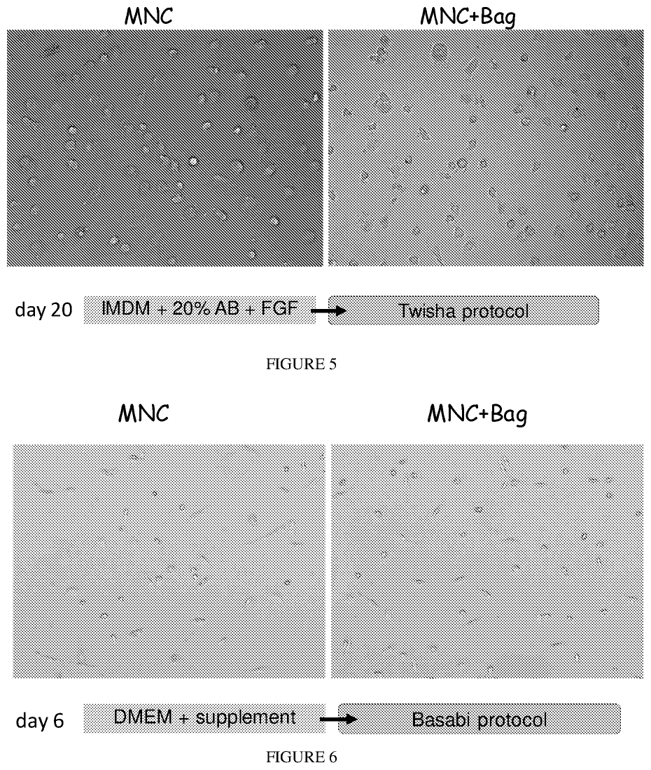

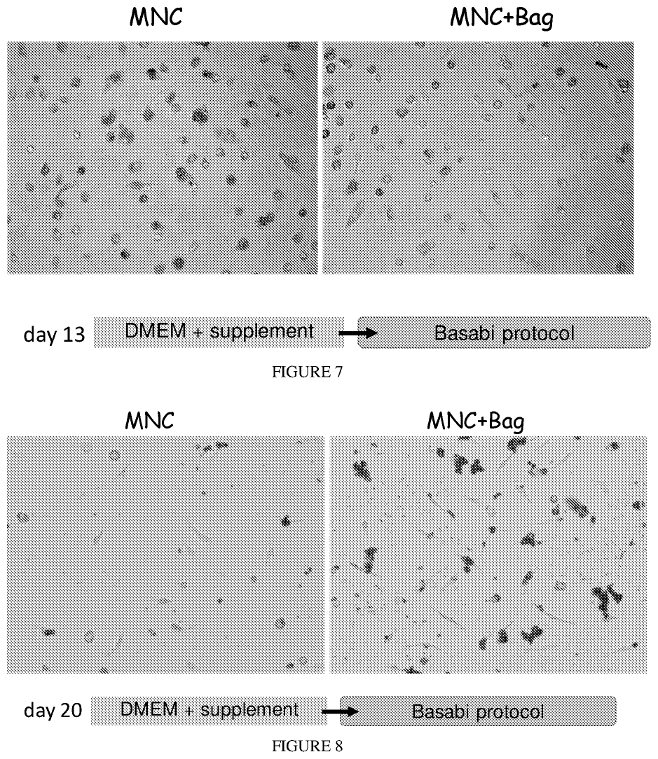

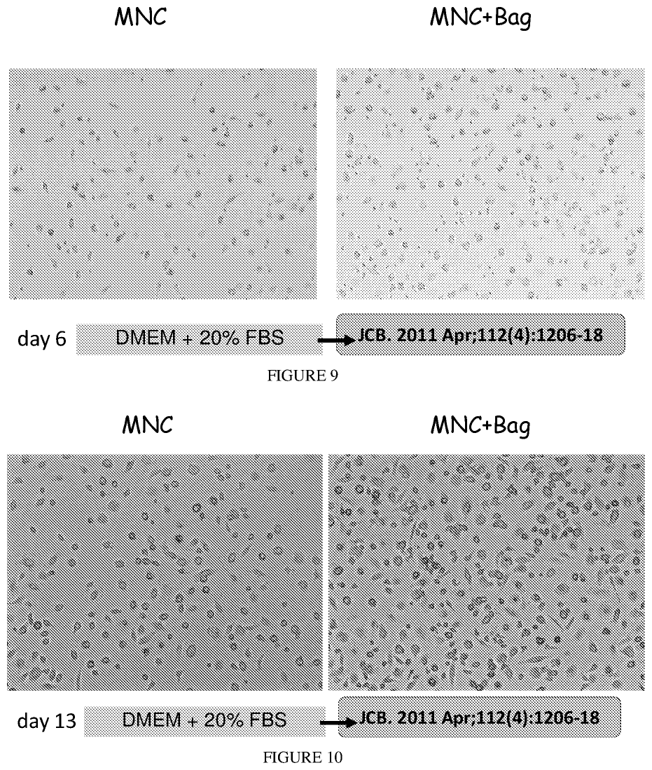

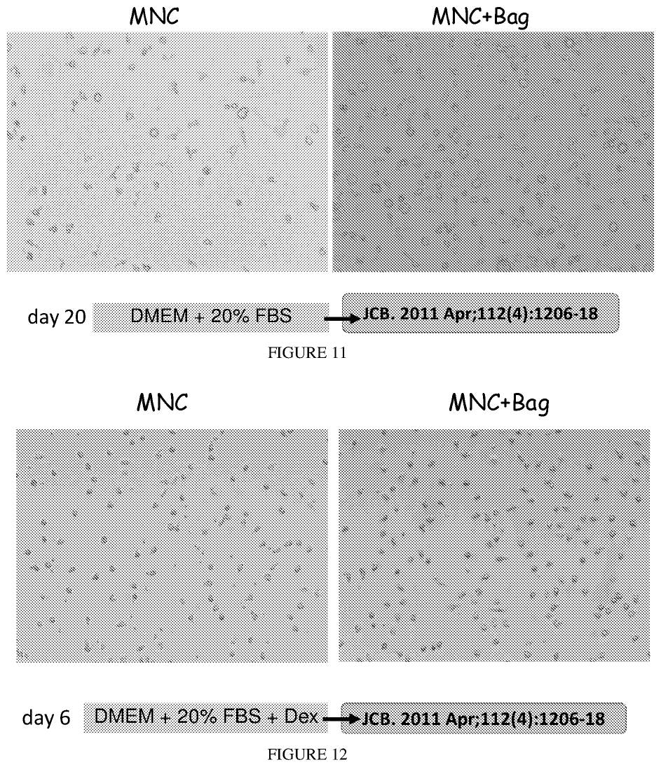

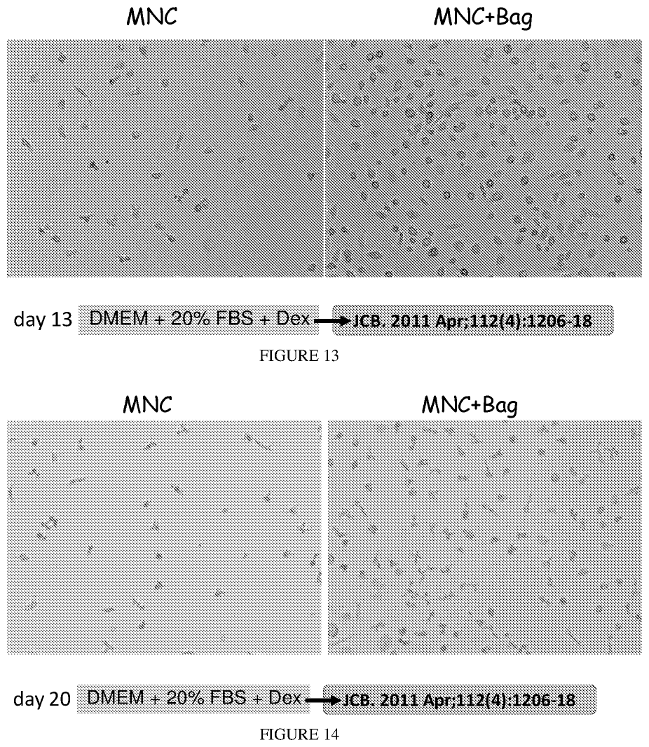

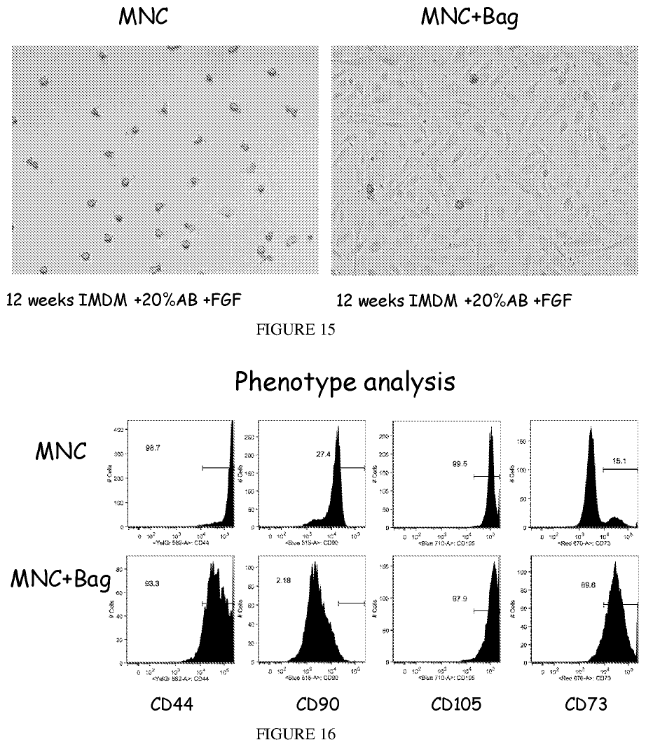

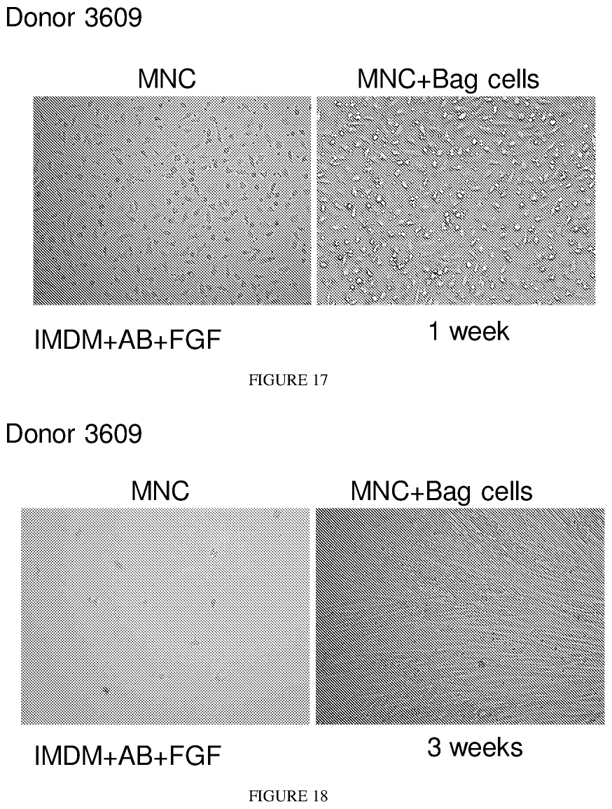

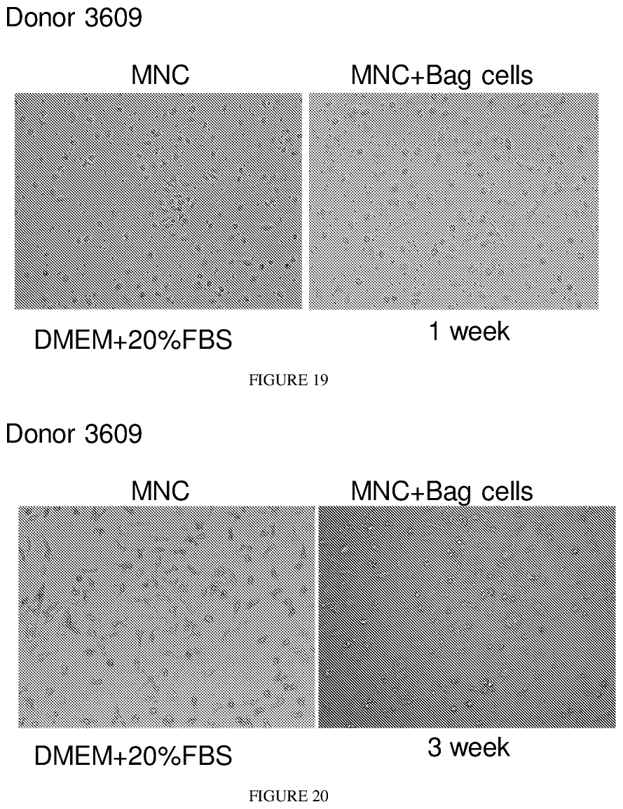

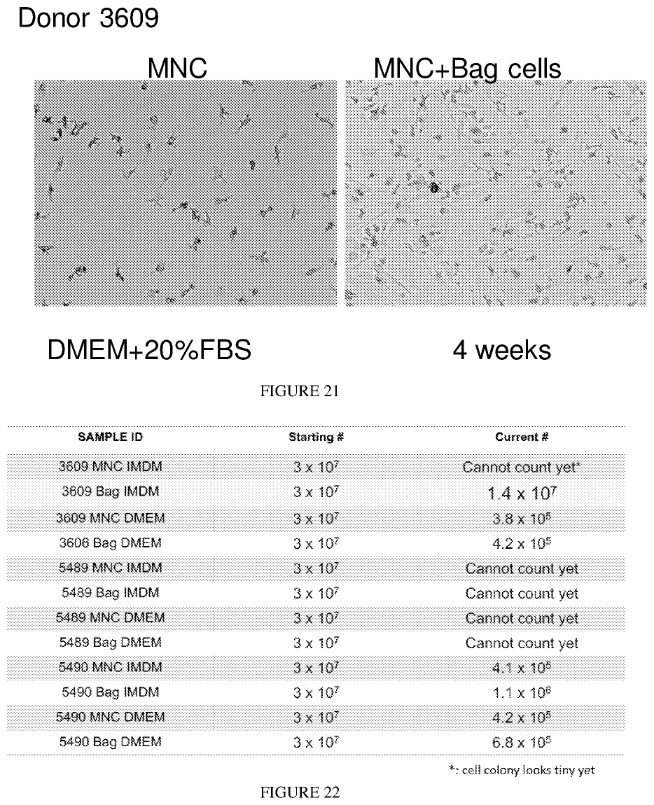

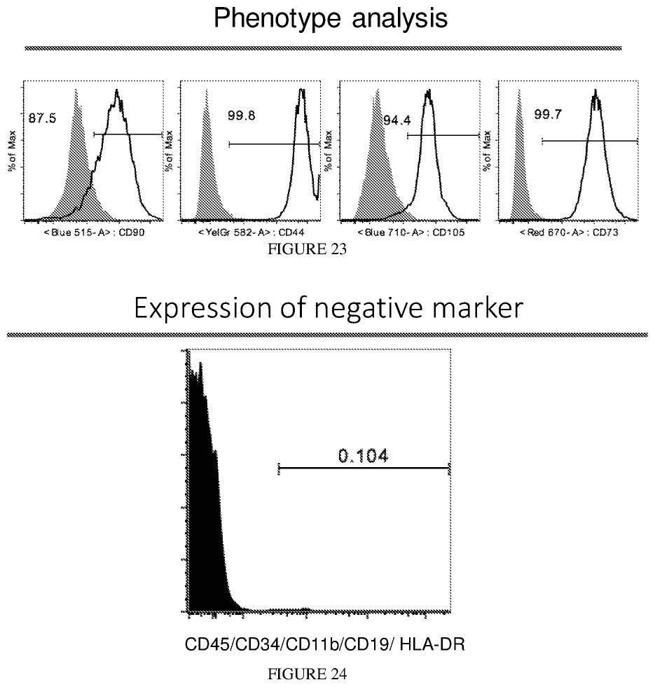

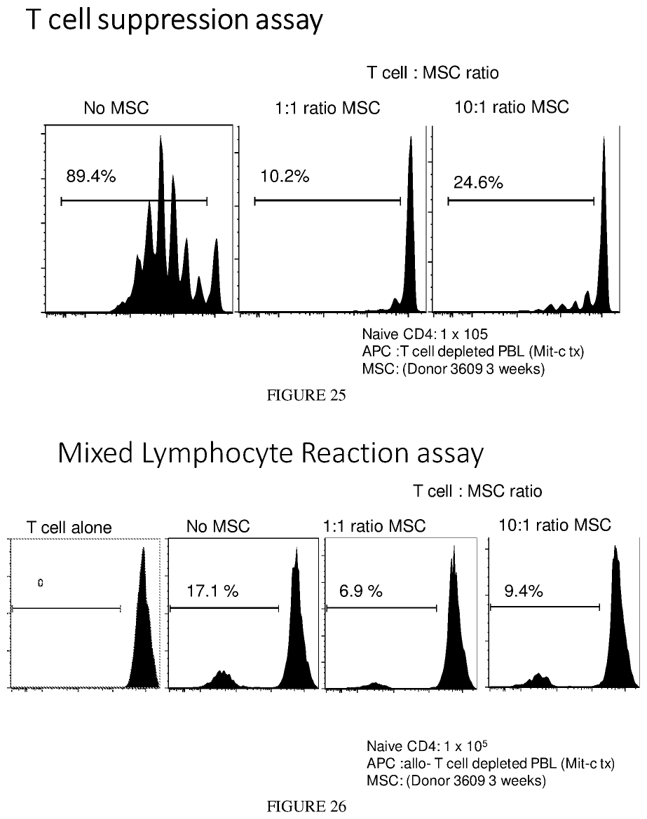

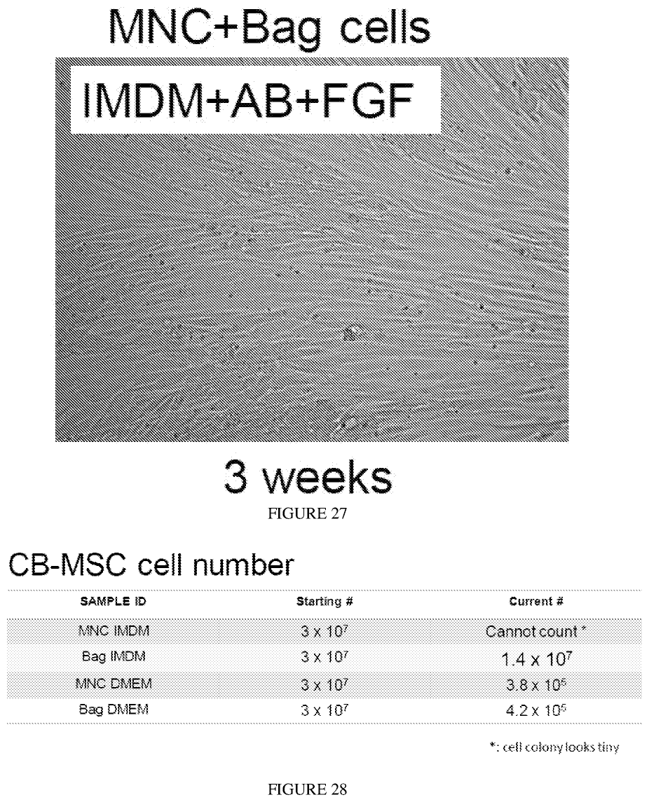

### DETAILED DESCRIPTION

The practice of the present invention will employ, unless otherwise indicated, conventional techniques of cellular biology, cell culturing, biochemistry and immunology, which are known to and employable by those of ordinary skill in the art.

Methods are provided for isolating mesenchymal stromal cells from umbilical cord blood. The present methods stem from the surprising finding that, when raw umbilical cord blood is disposed in a blood collection bag, mesenchymal stromal cells in the raw umbilical cord blood tend to precipitate out of the blood and adhere to the blood collection bag surface. Unlike prior methods that attempted to harvest mesenchymal stromal cells which remained suspended in raw umbilical cord blood in the blood collection bag, the present methods harvest mesenchymal stromal cells which have precipitated out of raw umbilical cord blood and adhered to the blood collection bag surface, yielding a substantially increased success rate in isolating mesenchymal stromal cells from umbilical cord blood for use in research and therapy.

When introducing elements of the present invention or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

It is understood that aspects and embodiments of the invention described herein include “consisting” and/or “consisting essentially of” aspects and embodiments.

Throughout this disclosure, various aspects of this invention are presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used herein, “raw umbilical cord blood” means unprocessed umbilical cord blood. Collecting umbilical cord blood from the placenta or the attached umbilical cord, disposing umbilical cord blood into a blood collection container (e.g. a blood collection bag), and preserving and storing unadulterated umbilical cord blood (e.g. refrigeration, adding an anticoagulant, etc.) are not considered processing steps within the context of this disclosure.

Methods are provided for isolating mesenchymal stromal cells from umbilical cord blood. In embodiments, the methods comprise one or more of the following steps: providing a blood collection container having raw umbilical cord blood contained therein; draining the umbilical cord blood from the blood collection container; rinsing the blood collection container with a first rinse solution; detaching mesenchymal stromal cells adhered to the blood collection container with a cell detachment solution introduced into the blood collection container to form a mesenchymal stromal cell enriched fluid; voiding the mesenchymal stromal cell enriched fluid into a collection apparatus; removing the cell detachment solution from the mesenchymal stromal cell enriched fluid; preparing an enriched population of mesenchymal stromal cells for tissue culture; and plating the enriched population of mesenchymal stromal cells in a tissue culture apparatus.

Umbilical cord blood can be provided in any suitable at least partially polymer lined blood collection container referred to generally herein as a “collection bag.”

Typically, umbilical cord blood is collected from the umbilical cord after the delivery of placenta or while in utero and contained in a flexible polymer blood bag; however, it is understood that the cord blood may be provided in other suitable polymer containers of varying shapes, sizes, and rigidity such as centrifuge tubes, tissue culture plates, and the like. Standard blood bag systems for the collection of cord blood are well known in the art. The blood collection bag, or container, can contain an anticoagulant such as a citrate-phosphate-dextrose solution, citrate-phosphate-dextrose with adenine, sodium citrate solution, citrate-dextrose solution, or heparin. Containers suitable for use in the invention can be commercially available blood collection bags such as from Pall Corporation (Covina, Calif.) made of polyvinyl chloride and/or ethyl vinyl acetate, or containers made of any polymers to which MSCs adhere such as treated polystyrene, polyvinyl chloride, and/or ethyl vinyl acetate.

Once raw umbilical cord blood is placed in a suitable container, the umbilical cord blood can be used as part of a method for isolating mesenchymal stromal cells from umbilical cord blood. The umbilical cord blood can be preserved for a period of time, in either refrigerated or frozen states, and then used at a later date as part of a method for isolating mesenchymal stromal cells from umbilical cord blood. In embodiments, raw umbilical cord blood is processed for mesenchymal stromal cells within about 24-72 hours of collection, preferably within about 24-48 hours. In embodiments, raw umbilical cord blood is maintained at room temperature for no more than 3 days in a suitable container to allow optimal mesenchymal stromal cell extraction from raw cord blood, including cells that have adhered to the inside surface of the container prior to dissociating the cells from the surface.

In embodiments, raw umbilical cord blood is provided in a suitable polymer lined container. Provided that mesenchymal stromal cells in the raw umbilical cord blood have had sufficient time to adhere to the container surface, the raw cord blood is then drained from the container, leaving behind a substantially empty container having mesenchymal stromal cells contained therein and adhered to the inner container surface.

The drained container can then be washed with a rinse solution, but it need not be. The rinse solution can be, for example, a saline solution such as Lactated Ringer's solution, Acetated Ringer's solution, intravenous sugar solutions (e.g. 5% dextrose in normal saline, 10% dextrose in normal saline, 5% dextrose in half-normal saline, 10% dextrose in half-normal saline), phosphate buffered saline (PBS), TRIS-buffered saline (TBS), Hank's balanced salt solution (HBSS), Earle's balanced salt solution (EBSS), standard saline citrate (SSC), HEPES-buffered saline (HBS), Gey's balanced salt solution (GBSS), and the like.

The drained container can be rinsed once or it can be rinsed multiple times (e.g. 2, 3, 4, 5, 6, or more times). Generally, a single rinse cycle can include adding a rinse solution to the drained container, agitating the rinse solution in the container (e.g. rotating the container by hand, placing the container on a shaker, etc.), and draining or removing the rinse solution from the container. In some embodiments, the container goes through serial rinse cycles until no more blood is evident to the eye.

In embodiments, mesenchymal stromal cells adhered to the lumen of the container surface are then detached. This is typically accomplished by introducing a cell detachment solution into the container. The cell detachment solution dissociates adhered mesenchymal stromal cells from the container surface. Cell detachment solutions are well known in the art and commercially available, and can include an enzyme for dissociating cells, a divalent cation chelator (e.g. calcium chelator, magnesium chelator, etc.), or a combination thereof, to dissociate adherent cells. Exemplary dissociation enzymes include collagenase, trypsin, elastase, hyaluronidase, papain, pronase, and dispase (neutral protease). Exemplary divalent cation chelators include ethylenediaminetetraacetic acid (EDTA) and ethylene glycol-bis(β-aminoethyl ether)-N,N,N′,N′-tetraacetic acid (EGTA). In some embodiments, the cell detachment solution is ACCUTASE Dissociation Buffer (Sigma A6964-100 ML) (proteolytic and collagenolytic enzymes in Dulbecco's PBS (0.2 g/L KCl, 0.2 g/L KH2PO4, 8 g/L NaCl, and 1.15 g/L Na2HPO4) containing 0.5 mM EDTA·4Na and 3 mg/L Phenol Red). Generally, the cell detachment step can include adding a cell detachment solution to the container and agitating the cell detachment solution in the container (e.g. rotating the container by hand, placing the container on a shaker, etc.). While enzymatic and chemical methods are preferred for dissociating adherent mesenchymal stromal cells, the cells can also be dissociated by mechanical methods (e.g. scraping or scratching cells off of the container surface).

After cellular detachment, the dissociated mesenchymal stromal cells can be removed from the original container and placed into a collection apparatus although they need not be. The collection apparatus is another container such as a conical tube or other centrifugation tube that facilitates further processing of the mesenchymal stromal cells. For example, if the original container is a blood collection bag, the dissociated mesenchymal stromal cells suspended in cell detachment solution can be drained into a collection apparatus (e.g. a centrifugation tube) for further processing. However, if the original container is a centrifugation tube, then the dissociated mesenchymal stromal cells suspended in cell detachment solution may not need to be moved to a collection apparatus for further processing.

In embodiments, removing mesenchymal stromal cells adhered to the lumen of the original container surface may include one or more infusions of a cell detachment solution into the original container. Each cell detachment solution infusion typically includes adding a cell detachment solution to the original container, agitating the cell detachment solution in the container for a period of time, and draining the cell detachment solution containing dissociated mesenchymal stromal cells into a collection apparatus. In embodiments, removing mesenchymal stromal cells adhered to the lumen of the original container surface may also include one or more infusions of a rinse solution (e.g., a 1X PBS solution) into the original container following a cell detachment infusion. Each rinse solution infusion typically includes adding a rinse solution to the original container, agitating the rinse solution in the container for a period of time, and draining the rinse solution containing dissociated mesenchymal stromal cells into a collection apparatus. The rinse solution infusions may help ensure collection of all mesenchymal stromal cells in the bag (e.g. mesenchymal stromal cells left behind after a cell detachment solution infusion).

In embodiments, the dissociated mesenchymal stromal cells suspended in cell detachment solution and/or rinse solution (e.g. a mesenchymal stromal cell enriched fluid) are then processed to remove the cell detachment solution and/or rinse solution. This can be accomplished by, for example, centrifuging the mesenchymal stromal cell enriched fluid to form a pellet comprising an enriched population of mesenchymal stromal cells and a supernatant comprising cell detachment solution and/or rinse solution, and aspirating the supernatant.

In embodiments, the pellet comprising mesenchymal stromal cells is then prepared for tissue culture. The pellet can be prepared for tissue culture by, for example, taking one or more of the following steps: washing the cells with a rinse solution and suspending the enriched population of mesenchymal stromal cells in a culture medium.

The enriched population of mesenchymal stromal cells can be rinsed once or it can be rinsed multiple times (e.g. 2, 3, 4, 5, 6, or more times). Generally, a single rinse cycle can include adding a rinse solution to the container holding the mesenchymal stromal cells, agitating the rinse solution in the container (e.g. rotating the container by hand, placing the container on a shaker, etc.), and draining or removing the rinse solution from the container. The rinse solution can be, for example, a saline solution such as Lactated Ringer's solution, Acetated Ringer's solution, intravenous sugar solutions (e.g. 5% dextrose in normal saline, 10% dextrose in normal saline, 5% dextrose in half-normal saline, 10% dextrose in half-normal saline), phosphate buffered saline (PBS), TRIS-buffered saline (TBS), Hank's balanced salt solution (HBSS), Earle's balanced salt solution

(EBSS), standard saline citrate (SSC), HEPES-buffered saline (HBS), Gey's balanced salt solution (GBSS), and the like.

In embodiments, the enriched population of mesenchymal stromal cells are suspended in a culture medium. Suitable culture mediums are well known in the art and can include, for example, RPMI1640, Ham's F10 medium, Ham's F12 medium, Mesenchymal Stem Cell Growth Medium, Iscove's modified Dulbecco's medium (IMDM), Dulbecco's modified Eagle's Medium (DMEM), advanced DMEM (Gibco), DMEM/MCDB201 (Sigma), CELL-GRO FREE, DMEM/F12, Eagle's basal medium, and the like. In some embodiments, the culture medium is supplemented with 0% to 15% (v/v) serum, glucose, an antibiotic agent, and/or an antimycotic agent.

In one preferred embodiment, the culture medium is Dulbecco's Modified Eagle's Medium (DMEM) with 20% Fetal Bovine Serum (FBS), with or without dexamethasone (DEX). In another embodiment, the culture medium is Iscove's Modified Dulbecco's Medium (IMDM) with 20% human serum and fibroblast growth factor (FGF). In another embodiment, the culture medium is Dulbecco's Modified Eagle's Medium (DMEM) with supplement (supplement is a human platelet rich plasma that serves as a cytokine rich replacement for fetal bovine serum, sold by Compass-Biomedical).

In embodiments, the enriched population of mesenchymal stromal cells suspended in culture medium is plated in a tissue culture apparatus.

In embodiments, mesenchymal stromal cells isolated in accordance with this disclosure will express surface proteins CD105+, CD90+, and CD73+, and will not express surface proteins CD34, CD14, CD31, CD11b, and HLA-DR. In embodiments, mesenchymal stromal cells isolated in accordance with this disclosure adhere to plastic surfaces such as tissue culture flask surfaces and blood collection bag surfaces. In embodiments, mesenchymal stromal cells isolated in accordance with this disclosure and plated on a tissue culture apparatus, such as a tissue culture flask, will display a spindle-shaped morphology.

In embodiments, the present methods for isolating mesenchymal stromal cells are performed under sterile conditions.

In embodiments, mesenchymal stromal cells isolated in accordance with the present methods can be cryopreserved for future use.

In embodiments, mesenchymal stromal cells isolated in accordance with the present methods (e.g., Bag Cells) can be co-cultured with mononuclear cells isolated by other techniques. In embodiments, mesenchymal stromal cells isolated in accordance with the present methods (e.g. Bag Cells) are co-cultured with mononuclear cells isolated from umbilical cord blood. In embodiments, mononuclear cells are isolated from umbilical cord blood drained from a blood collection bag and Bag Cells are obtained in accordance with the present methods by harvesting mesenchymal stromal cells adherent to the blood collection bag after draining the umbilical cord blood. Mononuclear cells may be isolated from umbilical cord blood drained from a blood collection bag by, for example, density gradient separation with Ficoll-Paque.

### EXAMPLES

The present invention is further illustrated by the following examples, which are not to be construed in any way as imposing limitations upon the scope thereof.

On the contrary, it is to be clearly understood that resort may be had to various other aspects, embodiments, modifications, and equivalents thereof which, after reading the description herein, may suggest themselves to one of ordinary skill in the art without departing from the spirit of the present invention or the scope of the appended claims. Thus, other aspects of this invention will be apparent to those skilled in the art from consideration of the specification and practice of the invention disclosed herein.

### Example 1

### Protocol for Detaching and Isolating Mesenchymal Stromal Cells Attached to the Walls of a Cord Blood Bag

Mesenchymal stromal cells were detached and isolated from the walls of a cord blood bag according to the following procedure:
1. Obtain a cord blood unit (e.g cord blood in a cord blood bag). Preferably, the cord blood is <24 hours old and the cord blood unit contains >80 mL of blood.
2. Drain cord blood from the cord blood bag.
3. After blood is drained from the cord blood bag, add 150 mL of Accutase Dissociation Buffer (Sigma A6964-100 ML) to the cord blood bag with a 30 mL syringe. Place the tip of the syringe inside of the cord blood bag tubing and pour fluid in.
4. After 150 mL of Accutase buffer has been added, wrap Parafilm around the end of the tubing of the cord blood bag (to ensure that no liquid leaves the bag)
5. Place cord blood bag into a sterile transport bin and then place on a shaker 1000 rpm for 10 minutes. This process is done at room temperature.
6. Drain the cord blood bag into 50 mL Falcon tubes.
7. Repeat infusion of Accutase Dissociation Buffer into the cord blood bag, shaking for 10 min and draining. Follow with two PBS (CCF Media Lab 121-1000p, Lot# 262) rinses (100 mL) of the cord blood bag to ensure collection of all cells.
8. Spin down the Falcon tubes containing the PBS rinse and also Accutase Dissociaton Buffer at 1200 rpm for 8 minutes.
9. Aspirate liquid and combine cells into a single 50 mL Falcon tube.
10. Wash these cells in PBS and spin down at 1200 rpm for 8 minutes.
11. Suspend the cell pellet in DMEM (CCF Media lab, cat# 18-500p) and 20% FBS (CCF Media lab).
12. Plate these cells in DMEM with 20% FBS in an appropriate sized flask and concentration to maintain 2×10⁶ cells/cm² with depth.
13. Add media to the flask after 72-120 hours. Replace media after 120-168 hours.

### Example 2

### Protocol for Isolating Mononuclear Cells from Cord Blood

Mononuclear cells were isolated from cord blood according to the following procedure:
1. Obtain a cord blood unit (e.g cord blood in a cord blood bag). Preferably, the cord blood is <24 hours old and the cord blood unit contains >80 mL of blood.
2. Drain cord blood from the cord blood bag into 50 mL falcon tubes.
3. Dilute 25 mL of blood with 25 mL of PBS (1:1 blood to PBS dilution).
4. After dilution, turn tubes upside down 3-4 times to mix together.
5. Pipet 15 mL of Ficoll-Paque density gradient medium (Ge Healthcare 17-1440-03 Lot # 10241256) into a 50 mL Sepmate Tube. Place the pipet tip against the vertex near the bottom of the tube. Slowly fill bottom of tube, make sure no bubbles are present.
6. Very slowly pipet 25 mL of the diluted blood-PBS mixture into the 50 mL Sepmate tube that contains the Ficoll. Place the pipette tip against the side of the tube and slowly release the mixture into the tube, making sure that no blood enters the bottom portion of the sepmate tube. Keep Sepmate tube vertical.
7. Centrifuge (at 2400 rpm (1200xg) for 40 minutes with the brake on.
8. After centrifugation (RBCs remain on the bottom) the top layer is pipetted off. The top layer contains the buffy coat, along with the plasma. Combine this fraction with layers from other tubes into a 50 mL falcon tube.
9. Centrifuge these tubes at 1200 rpm for 8 minutes.
10. Aspirate liquid leaving the pellet at the bottom of the tube.
11. Wash MNCs with 1X PBS (add up to 50 mL) and spin down at 1200 rpm for 8 minutes.
12. Aspirate liquid and wash MNCs again with 1X PBS and spun down at 1200 rpm for 8 minutes.
13. Aspirate liquid
14. Depending on nucleated red blood cell concentration, ACK lysing buffer (Gibco A10492-01) can be used to lyse RBC. Pipet 5 mL of buffer to suspend the pellet. After 5 minutes, spin down the tubes at 1200 rpm for 8 minutes.
15. Aspirate liquid.
16. Wash cells with 1X PBS and spin down at 1200 rpm for 8 minutes.
17. Aspirate liquid.
18. Suspend cell pellet with 10 mL of DMEM with 20% FBS.
19. Plate these cells in DMEM with 20% FBS in an appropriate sized flask and concentration to maintain 2×10⁶ cells/cm² with depth.

### Example 3

### Protocols for Depleting CD133+ Cells

Protocol for depleting CD133+ mononuclear cells isolated from cord blood:
1. Obtain suspension of mononuclear cells isolated according to example 2 above.
2. Centrifuge at 1500 rpm for 5 min.
3. Aspirate liquid leaving the pellet at the bottom of the tube.
4. Suspend the cell pellet in an isolation buffer (1X PBS, 0.5% Human Serum, 2 mM EDTA). Add 100 μl of FcR Blocking Reagent per 10⁸ cells and 100 μl of CD133 MicroBeads per 10⁸ cells (CD133+ Microbead Kit: Miltenyi (Catalog number: 130-100-830)). Mix well using a vortex and incubate at 4-8° C. for 30 mins.
5. Wash cells by adding 1-2 mL of isolation buffer per 10⁸ cells and centrifuge for 5 min at 1500 rpm.
6. Aspirate supernatant completely.
7. Resuspend cell pellet with 500 μL isolation buffer.
8. Meanwhile, prepare the Magnetic-Activated Cell Sorting (MACS) separator for the magnetic separation.
9. Prepare each LS column by rinsing with 3 ml isolation buffer. Discard flow-through.
10. Apply cell suspension onto the prepared LS column
11. Wash each LS column three times with 3 ml isolation buffer per column. Only add new buffer when the column reservoir is empty.
12. The collected total effluent contains the unlabelled CD133− cell fraction.
13. Remove the LS column from the separator and place it on a new collection tube.
14. Apply 5 ml of isolation buffer onto the LS column Immediately flush out the labelled CD133+ cells by firmly pushing the plunger into the column.
15. Plate the CD133− cells in DMEM with 20% FBS in an appropriate sized flask and concentration to maintain 2×10⁶ cells/cm² with depth.

Alternative protocol for removing CD133+ hematopoietic stem cells isolated from cord blood by cell sorting using PE conjugated CD133 antibody:
1. Obtain suspension of mononuclear cells isolated according to example 2 above.
2. Resuspend cells in a surface staining buffer (0.1% BSA in PBS).
3. Stain cells with a PE-CD133 antibody (clone W6B3C1).
4. Incubate cells for 20 minute at 4° C. degree, and wash twice with staining buffer.
5. Sort CD133− cells using FACS.
6. Plate sorted CD133 cells in an appropriately sized flask.

### Example 4

### Protocol for Mixed Lymphocyte Reaction (MLR) Assay to Test UCB Derived MSC Suppressive Function

Protocol for isolation of AB naïve CD4 T cells (A) and T cell depleted PBL (B) from AB blood samples
1. Add density gradient medium (GE Healthcare, Cat#: 17-1440-03) to the 50 ml

SepMate™ tube (STEMCELL technologies, Cat#: REF 85450) by carefully pipetting it through the central hole of the SepMate™ insert. The top of the density gradient medium will be above the insert.
2. Dilute AB sample (A) with an equal volume of PBS (PBS 1x w/o Ca, w/o Mg; CCF Media Lab 121-1000p, Lot# 262)+2% FBS (CCF Media Lab FBS-500 HI). Mix gently.
3. Keeping the SepMate™ tube vertical, add the diluted sample by pipetting it down the side of the tube (no more than 17 ml per tube). The sample will mix with the density gradient medium above the insert.
4. Centrifuge at 1200×g for 15 minutes at room temperature, with the brake on.
5. Pour off the top layer, which contains the enriched MNCs, into a new tube. Do not hold the SepMate™ tube in the inverted position for longer than 2 seconds. That will allow to separate MNC fraction from red blood cells. Spin down MNC fraction at 1000 rpm, 15 min. RT. Resuspend the cell pellet.
6. Wash enriched MNCs with PBS+2% FBS. Repeat wash
7. Count cells in hemocytometer with Trypan-Blue Stain (Sigma)
8. Determine cell number and Centrifuge cell suspension at 1,000 rpm for 10 minutes. Aspirate supernatant completely.
9. Resuspend cell pellet in 40 μl of binding buffer (PBS-2% BSA-2 mM EDTA) per 5×10⁷ total cells in 5 ml tube.
10. Add 10 μl/ml Biotinylated Naïve CD4 T cell cocktail II antibody (Human Naïve CD4 T cell isolation kit II, Miltenyi biotech, Cat# 130-094-131) to sample and incubate for 5 min in (2-8 degrees)
11. Add 30 ul of binding buffer and add 20 ul of Naïve CD4 T cell Microbeads Cocktail II (Miltenyi biotech, Cat# 130-045-101) to sample incubate at 2-8 degrees for 10 min.
12. Add binding buffer up to 0.5 ml
13. Place the tube into the Auto MACS
14. Count collected naïve CD4 T cells and perform a CFSE stain (ThermoFisher Scientific, Cat#C34554)
15. Wash Cells with PBS twice and resuspend cells in 20 million cells per ml in PBS
16. Prepare of 1 uM CFSE in PBS and incubate 8 min at RT at dark place
17. Add same volume of FBS into cells and incubate 1 min to stop CFSE reaction
18. Fill the tube with complete RPMI and spin down twice
19. For T cell stimulation, remove T cells by AB (B) with microbeads conjugated Human CD4 and CD8 reagent (Human CD4 Microbeads, Miltenyi biotech, Cat# 130-045-101; Human CD8 Microbeads, Miltenyi biotech, Cat# 130-045-201)
20. Run Auto MACS on human CD4 and CD8 microbeads stained PB MNCs.
21. Treat collected 1×10⁸ PBL cells with 1 mg/ml Mitomycin C to inhibit proliferation (Toris™, Cat# 32-581-0)
22. Incubate cells for 20 min at 37 C and wash with media

Protocol for MLR Assay of UCB Derived MSC
1. 2×10⁵ CFSE labeled AB naïve CD4 T cells were seeded into 96 well U plate
2. To activate CFSE labeled CD4 T cells, 2×10⁵ Mitomycin treated T-depleted PBL cells were added into T cell added well
3. MSCs were added into CFSE labeled naïve CD4 T cells at various ratios (naïve CD4:MSC ratio; 1:0, 1:1, 10:1, and 100:1 etc.) and incubated.
4. Cells were harvested and surface CD4 stained at day 5 post stimulation
5. Analyze samples by flow cytometry

### Example 5

### Protocol for UCB Derived MSC FACS Phenotyping

Protocol for deriving and characterizing MSC obtained from umbilical cord blood:
1. Obtain MSC cells from umbilical cord blood cells culture flask following the dissociation protocol (see example 1 above).
2. Spin down, aspirate supernatant, resuspend
3. Determine cell number by trypan blue stain (Sigma, cat# T8154)
4. Take nine aliquots each with up to 5×10⁵ MSCs per tube and wash cells with FACS buffer (0.1% BSA 1 mM EDTA in 1X PBS w/o Ca, w/o Mg (CCF Media Lab 121-1000p, Lot# 262))
5. Centrifuge cell suspension at 1200 rpm for 5 minutes.
6. Aspirate supernatant completely
7. Resuspend aliquots in 100 uL of FACS buffer, respectively
8. Resuspend aliquot blank in 500 uL of FACS buffer. This aliquot is ready-to-use. Proceed directly with compensation
9. Add appropriate amount of antibodies into tube. The MSC antibodies are from

BD Biosciences Human MSC Analysis Kit (Cat# 562245) and include: PE hMSC Negative Cocktail (CD34, CD45, CD11b, CD19 and HLA-DR); PE hMSC Isotype Control Negative Cocktail; hMSC Positive Cocktail; hMSC Isotype Control Positive Cocktail; FITC Mouse Anti-Human CD90; PE Mouse Anti-Human CD44; PE Mouse IgG2b, k Isotype Control; APC Mouse Anti-Human CD73; PerCP-Cy™5.5 Mouse Anti-Human CD105 (Endoglin); CD45 PE
10. Mix each aliquot well and incubate for 30 minutes in the dark in the refrigerator (4 degrees C.)
11. Wash each aliquot tube by adding 2 mL of FACS buffer and centrifuge 1200 rpm for 5 minutes. Aspirate supernatant completely.
12. Wash cells by adding 1-2 mL of buffer and centrifuge 1200 rpm for 5 minutes. Aspirate supernatant completely.
13. Resuspend each cell pellet separately in 300 uL FACS buffer
14. Analyze samples by FACS analysis

1. A method for isolating mesenchymal stromal cells from umbilical cord blood comprising:
providing a polymer-lined blood collection container having umbilical cord blood contained therein;
draining the umbilical cord blood from the blood collection container;
detaching mesenchymal stromal cells adhered to the blood collection container with a cell detachment solution introduced into the blood collection container to form a mesenchymal stromal cell enriched fluid; and
voiding the mesenchymal stromal cell enriched fluid into a collection apparatus.

2. The method of claim 1, further comprising:
rinsing the blood collection container with a first rinse solution after the draining step but prior to the detaching step.

3. The method of claim 2, wherein the rinsing step is repeated.

4. The method of claim 2, wherein the rinsing step comprises:
adding the first rinse solution to the blood collection container;
agitating the first rinse solution; and
removing the first rinse solution from the blood collection container.

5. The method of claim 2, wherein the first rinse solution is a saline solution.

6. The method of claim 1, further comprising:
removing the cell detachment solution from the mesenchymal stromal cell enriched fluid; and
preparing an enriched population of mesenchymal stromal cells for tissue culture.

7. The method of claim 1, wherein the cell detachment solution is agitated in the blood collection container during the detaching step.

8. The method of claim 1, wherein the cell detachment solution comprises a cell detachment enzyme.

9. The method of claim 1, wherein the cell detachment solution comprises trypsin.

10. The method of claim 1, wherein the cell detachment solution comprises a calcium chelator, a magnesium chelator, or a combination thereof.

11. The method of claim 1, wherein the cell detachment solution comprises ethylenediaminetetraacetic acid.

12. The method of claim 1, wherein the collection apparatus is a centrifugation tube.

13. The method of claim 6, wherein the removing step comprises:
centrifuging the mesenchymal stromal cell enriched fluid to form a first supernatant and a first pellet comprising mesenchymal stromal cells; and
aspirating the first supernatant.

14. The method of claim 13, wherein the preparing step comprises:
suspending the first pellet in a second rinse solution to form a rinsed mesenchymal stromal cell solution;
spinning down the rinsed mesenchymal stromal cell solution to form a second supernatant and a second pellet comprising mesenchymal stromal cells;
aspirating the second supernatant; and
suspending the second pellet in a culture medium.

15. The method of claim 14, wherein the culture medium comprises Dulbecco's Modified Eagle's Medium with 20% fetal bovine serum.

16. The method of claim 14, wherein the culture medium comprises Dulbecco's Modified Eagle's Medium with 20% fetal bovine serum and dexamethasone

17. The method of claim 14, wherein the second rinse solution is a saline solution.

18. The method of claim 6, further comprising:
plating the enriched population of mesenchymal stromal cells in a tissue culture apparatus.

19. The method of claim 16, wherein the plated mesenchymal stromal cells are plastic adherent and display a spindle-shaped morphology.

20. The method of claim 6, wherein the mesenchymal stromal cells in the enriched population of mesenchymal stromal cells express surface proteins CD105+, CD90+, and CD73+, and wherein the mesenchymal stromal cells in the enriched population of mesenchymal stromal cells do not express surface proteins CD34, CD14, CD31, CD11b, and HLA-DR.

21. The method of claim 18, wherein the enriched population of mesenchymal stromal cells are co-cultured with mononuclear cells.
